(19) United States
(12) Patent Application Publication
(10) Pub. No.: US 2017/0037091 A1
(43) Pub. Date: Feb. 9, 2017

(54) STY PEPTIDES FOR INHIBITION OF ANGIOGENESIS

(71) Applicant: RESEARCH DEVELOPMENT FOUNDATION, Carson City, NV (US)

(72) Inventors: Tim STOUT, Houston, TX (US); Bissan AHMED, Portland, OR (US); Binoy APPUKUTTAN, Adelaide (AU); Trevor J. McFARLAND, Portland, OR (US)

(73) Assignee: Research Development Foundation, Carson City, NV (US)

(21) Appl. No.: 15/121,164

(22) PCT Filed: Feb. 25, 2015
(86) PCT No.: PCT/US2015/017522

§ 371 Date: Aug. 24, 2016

Related U.S. Application Data
(60) Provisional application No. 61/944,274, filed on Feb. 25, 2014.

(51) Int. Cl. C07K 14/47; A61K 45/06; A61K 38/17

(52) U.S. Cl. CPC C07K 14/4703; A61K 38/1709; A61K 45/06; A61K 38/00

(57) ABSTRACT

Peptides for the treatment of cancer and angiogenic disorders are provided. In some aspects, the peptides may be used to decrease angiogenesis or VEGF expression or function in a cancer such as, e.g., an ocular cancer. In some embodiments, a peptide of the present invention may be used to treat an angiogenic eye disorder such as, e.g., diabetic retinopathy.

This application claims the benefit of United States Provisional Patent Application No. 61/944,274, filed Feb. 25, 2014, the entirety of which is incorporated herein by reference.

### BACKGROUND OF THE INVENTION

1. Field of the Invention

The present invention relates generally to the fields of molecular biology and medicine. More particularly, it concerns peptides that can be used to inhibit angiogenesis and/or treat cancer.

2. Description of Related Art

Solid tumors depend on the formation of new blood vessels from preexisting vessels to supply them with nutrients and oxygen in order to grow beyond a size of 1-2 mm³ (Folkman, 2007). In addition to the need for an expanding vascular network, evidence suggests that some tumor cell proliferation can be directly influenced by VEGF auto-regulation. Angiogenesis is a complex multistep process that starts with vascular endothelial growth factor (VEGF)-induced vasodilatation and increased vascular permeability of pre-existing capillaries or post-capillary venules.

Transcriptional enhancer factor 1-related (RTEF-1) is present within ocular vascular endothelial cells and plays a role in the control of the transcription of the VEGF gene (Appukuttan et al., 2007). Full-length RTEF-1 is known to stimulate cell proliferation in vitro. In cancer biology, internal hypoxic conditions are a common feature of solid tumors (Cavazzoni et al., 2013; Xia et al., 2012; Ramaekers et al., 2011), genes associated with cancer metastasis are upregulated under hypoxic conditions, and hypoxic gene signatures are associated with poor prognosis (Toustrup et al., 2012). Hypoxia-induced genes, such as VEGF-A, initiate tumor vascularization (Ferrara, 2005). Related transcriptional enhancer factor-1 (RTEF-1) plays an important role in transcriptional regulation of angiogenic genes in hypoxic endothelial cells and independent factor of HIF-1 alpha (Zhang et al., 2009). However, other studies suggest that RTEF-1, acting via HIF-1, is a key regulator of angiogenesis in response to hypoxia (Zhang et al., 2009). As cancer continues to be a significant problem, clearly there is a need for new methods of modulating angiogenesis, e.g., for the treatment of cancer.

### SUMMARY OF THE INVENTION

The present invention, in certain aspects, provides peptides that may be used to inhibit angiogenesis, e.g., for the treatment of cancer or ocular neovascularization.

An aspect of the present invention relates to a peptide comprising a region having an amino acid sequence at least 90% identical to SEQ ID NO:1, wherein the peptide does not comprise a full-length RTEF-1 polypeptide, an RTEF 669 isoform (SEQ ID NO:20), an RTEF 651 isoform (SEQ ID NO:21), or an RTEF 366 isoform (SEQ ID NO:22); and wherein the peptide can reduce VEGF promoter activity. The peptide may comprise less than 121 contiguous amino acids of an RTEF-1 polypeptide. The peptide may be less than 121 amino acids in length. In some embodiments, the peptide comprises no more than 45 contiguous amino acids of an RTEF-1 polypeptide. The peptide may be less than 45 amino acids in length. The peptide may comprise an amino acid sequence at least 95% identical or at least 97% identical to SEQ ID NO:1. In some embodiments, the peptide comprises the sequence of SEQ ID NO:1. The peptide may be conjugated or fused to a cell importation signal sequence. In some embodiments, the peptide is fused or covalently coupled to a cell targeting moiety and/or a linker such as, e.g., a linker that is cleavable in a cancerous cell. In some embodiments, the peptide is covalently coupled to a cell importation signal sequence. The cell importation signal sequence may be the sequence of any one of SEQ ID NOs:4-19. In some embodiments, the cell importation signal sequence is the sequence of SEQ ID NO:4. The peptide may comprise STY-RMR (SEQ ID NO:2). In some embodiments, the peptide consists of STY-RMR (SEQ ID NO:2). The peptide may be a synthetic peptide or a recombinant peptide. In some embodiments, the peptide is 25-45, 26-40, or 26-36 amino acids in length. In some embodiments, the peptide is or consists of SEQ ID NO: 1. The peptide may be comprised in a pharmaceutical composition. The pharmaceutical composition may be formulated for intravenous, intratumoral, parenteral, intraocular, intracorneal, or intravitreal administration.

Another aspect of the present invention relates to a fusion protein comprising: (i) a peptide comprising a region that is at least 90% identical to SEQ ID NO:1, wherein the peptide does not comprise a full-length RTEF-1 polypeptide, an RTEF 669 isoform (SEQ ID NO:20), an RTEF 651 isoform (SEQ ID NO:21), or an RTEF 366 isoform (SEQ ID NO:22); and (ii) a heterologous amino acid sequence; wherein the fusion protein can reduce VEGF promoter activity. The peptide may comprises less than 121 contiguous amino acids of an RTEF-1 polypeptide. The peptide may comprise no more than 45 contiguous amino acids of an RTEF-1 polypeptide. The peptide may be less than 121 amino acids in length. In some embodiments, the peptide is less than 45 amino acids in length. In some embodiments, the fusion protein is less than 45 amino acids in length. The peptide may have an amino acid sequence at least 95% identical or at least 97% identical to SEQ ID NO:1. The heterologous amino acid sequence may be a cell importation signal sequence. The heterologous amino acid may be a cell targeting moiety and/or a linker such as, e.g., a linker that is cleavable in a cancerous cell. In some embodiments, the cell importation signal sequence is RMR (SEQ ID NO:4). The fusion protein may comprise or consist of STY-RMR (SEQ ID NO:2). The peptide may be comprised in a pharmaceutical composition.

Yet another aspect of the present invention relates to a composition comprising a peptide comprising a region that is at least 90% identical to SEQ ID NO:1, wherein the peptide does not comprise a full-length RTEF-1 polypeptide, an RTEF 669 isoform (SEQ ID NO:20), an RTEF 651 isoform (SEQ ID NO:21), or an RTEF 366 isoform (SEQ ID NO:22); and wherein the peptide is chemically conjugated to a heterologous amino acid sequence; wherein the composition can reduce VEGF promoter activity. The peptide may comprise less than 121 contiguous amino acids of an RTEF-1 polypeptide. The peptide may comprise no more than 45 contiguous amino acids of an RTEF-1 polypeptide. The peptide may be less than 121 amino acids in length. The peptide may be less than 45 amino acids in length. The peptide may have an amino acid sequence at least 95% or at least 97% identical to SEQ ID NO:1. In some embodiments, the peptide is or consists of the sequence of SEQ ID NO:1. The heterologous amino acid sequence may be a cell importation signal sequence. The heterologous amino acid may be a cell targeting moiety and/or a linker such as, e.g., a linker that is cleavable in a cancerous cell. In some embodiments, the cell importation signal sequence is RMR (SEQ ID NO:4). The peptide may comprise or consist of STY-RMR (SEQ ID NO:2). In some embodiments, the composition is a pharmaceutical composition comprising an excipient.

Another aspect of the present invention relates to a nucleic acid comprising a nucleic acid segment encoding a peptide or fusion protein of the present invention. The nucleic acid may be comprised in a vector. The vector may be a viral vector or a liposome. The vector may be a viral vector that is an adenovirus vector, an adeno-associated virus vector, a herpes virus vector, an SV-40 virus vector, a retrovirus vector, or a vaccinia virus vector. The nucleic acid segment may be operatively linked or coupled to a promoter. The promoter may be a cell type specific promoter or an inducible promoter. The inducible promoter may be a hypoxia inducible promoter. The inducible promoter may be an angiogenesis inducible promoter. The nucleic acid may encode two or more antiangiogenesis proteins.

Yet another aspect of the present invention relates to a cell comprising a nucleic acid of the present invention. The cell may be a bacterium, a yeast, an insect cell, or a mammalian cell.

Another aspect of the present invention relates to a viral vector comprising a nucleic acid of the present invention. The viral vector may be a lentivirus, an adenovirus, or an adeno-associated virus.

Yet another aspect of the present invention relates to a method of producing a peptide of the present invention comprising: a) expressing a nucleic acid of the present invention in a cell; and b) collecting the peptide or fusion protein therefrom.

Another aspect of the present invention relates to a method of decreasing angiogenesis in an organism comprising administering to the organism a peptide, fusion protein, or composition of the present invention. The organism may be a mammal such as, e.g., a human.

Yet another aspect of the present invention relates to a method of treating a cancer or an angiogenic eye disease in a mammalian subject, comprising administering to the subject a therapeutically effective amount of a peptide, fusion protein, or composition of any one of the present invention. The subject may be a human, mouse, rat, primate, monkey, or ape. In some embodiments, the subject is a human. The subject may have a cancer. The cancer may be a breast cancer, a retinoblastoma, a melanoma, or an ocular cancer. The cancer may be an ocular cancer selected from the group consisting of an ocular metastasis, an ocular micro-metastasis, or an ocular melanoma.

Another aspect of the present invention relates to a pharmaceutical composition comprising: a peptide, fusion protein, or composition of the present invention; and a pharmaceutically acceptable carrier or excipient. The pharmaceutical composition may be formulated for intravenous, intratumoral, parenteral, intraocular, intracorneal, or intravitreal administration.

Yet another aspect of the present invention relates to a pharmaceutical composition of the present invention for treating a subject with a disorder associated with abnormal cell growth or abnormal cell proliferation. The disorder associated with abnormal cell growth or abnormal cell proliferation may be an angiogenic disorder, a cancer, ocular neovascularization, an arterio-venous malformation, coronary restenosis, peripheral vessel restenosis, glomerulonephritis, or rheumatoid arthritis. In some embodiments, the angiogenic disorder is cancer. The cancer may be breast cancer, lung cancer, prostate cancer, leukemia, lymphoma, head and neck cancer, brain cancer, stomach cancer, intestinal cancer, colorectal cancer, renal cancer, bladder cancer, testicular cancer, esophageal cancer, ocular melanoma, retinoblastoma, liver cancer, ovarian cancer, skin cancer, cancer of the tongue, cancer of the mouth, or metastatic cancer. In some embodiments, the angiogenic disorder is ocular neovascularization. The ocular neovascularization may be neovascularization due to age-related macular degeneration, neovascularization due to corneal graft rejection, neovascularization due to retinopathy of prematurity (ROP), or neovascularization due to diabetic retinopathy. The subject may be further treated with an additional therapy for the disorder. The additional therapy may be an antibody that binds to VEGF, a VEGF receptor, FGF, an FGF receptor, bevacizumab, ranibizumab, or pegaptanib sodium. The additional therapy may be an anticancer therapy that is chemotherapy, surgical therapy, immunotherapy or radiation therapy. In some embodiments, the subject is a human. The composition may be administered intravenously, intraarterially, epidurally, intrathecally, intraperitoneally, subcutaneously, orally, or topically. The composition may be administered locally to the eye by topical drops, intracameral injection, subconjunctival injection, subtenon injection, or by intravitreous injection.

Another aspect of the present invention relates to use of a peptide, fusion protein, or composition of the present invention in the manufacture of a medicament for the treatment of a disorder associated with abnormal cell growth or abnormal cell proliferation.

Yet another aspect of the present invention relates to a method of treating a disorder associated with abnormal cell growth or abnormal cell proliferation in a subject in need thereof comprising administering a therapeutically effective amount of the pharmaceutical composition of the present invention to the subject.

Another aspect of the present invention relates to a kit comprising a predetermined quantity of a peptide, fusion protein, or composition of the present invention or a nucleic acid of the present invention in one or more sealed vials.

In a particular embodiment, the disorder is cancer. Non-limiting examples of cancer include cancer of breast cancer, lung cancer, prostate cancer, leukemia, lymphoma, head and neck cancer, brain cancer, stomach cancer, intestinal cancer, colorectal cancer, renal cancer, bladder cancer, testicular cancer, esophageal cancer, ocular melanoma, retinoblastoma, liver cancer, ovarian cancer, skin cancer, cancer of the tongue, cancer of the mouth, or metastatic cancer.

In some embodiments, the angiogenic disorder is ocular neovascularization. Non-limiting examples of ocular neovascularization include neovascularization due to age-related macular degeneration, neovascularization due to corneal graft rejection, neovascularization due to retinopathy of prematurity (ROP), or neovascularization due to diabetic retinopathy.

The methods of the present invention may further involve administering to the subject one or more secondary therapies for treatment of a disorder. For example, the secondary therapy may be a secondary therapy of an angiogenic disorder, a disorder associated with abnormal cell growth, a disorder associated with abnormal organ growth, or a disorder associated with impaired cell contact inhibition, or a disorder associated with increased YAP activity.

In some aspects, peptides are provided herein that can inhibit angiogenesis, but comprise an amino acid sequence that is shorter than an RTEF polypeptide. “RTEF polypeptide,” as used herein, refers to a full length RTEF-1 polypeptide (e.g., as described in U.S. 2009/0117119; expressed from NCBI Ref# NG_029958.1) or an alternatively spliced isoform RTEF-1 isoform such as RTEF isoforms 669, 651, and 339 (e.g., as described in U.S. 2012/0063994 or Appukuttan et al., 2007). The RTEF-1 polypeptide may have the sequence of: a full length RTEF polypeptide (SEQ ID NO:32), an RTEF isoform 669 (SEQ ID NO:20), RTEF isoform 651 (SEQ ID NO:21), or RTEF isoform 366 (SEQ ID NO:22). In some embodiments, the peptide may comprise less than 121, 120, 119, 118, 117, 116, 115, 110, 105, 100, 95, 90, 85, 80, 75, 70, 65, 60, 55, or less than or equal to 50, 49, 48, 47, 46, 45, 44, 43, 42, 41, 40, 39, 38, 37, 36, 35, 34, 33, 32, 31, 30, 29, 28, 27, 26, 25, or 24 contiguous amino acids of an RTEF-1 polypeptide.

In some embodiments, the secondary therapy is an antibody that binds to VEGF, a VEGF receptor, FGF, an FGF receptor, bevacizumab, ranibizumab, or pegaptanib sodium. The secondary therapy may be an anticancer therapy that is chemotherapy, surgical therapy, immunotherapy or radiation therapy. In particular embodiments, the subject is a mammal. Non-limiting examples of mammals include mice, rats, rabbits, dogs, cats, goats, sheep, horses, cows, primates, and humans. In specific embodiments, the subjects are humans.

Administration of the compositions set forth herein may be by any method known to those of ordinary skill in the art. Non-limiting examples of routes of administration include intravenously, intraarterially, epidurally, intrathecally, intraperitoneally, subcutaneously, orally, or topically. In some embodiments directed to the treatment or prevention of an ocular disorder, the composition is administered locally to the eye by topical drops, intracameral injection, subconjunctival injection, subtenon injection, or by intravitreous injection. Further detail concerning administration and dosage is discussed in the specification below.

Further aspects of the present invention concern kits that include a predetermined quantity of one or more peptides (e.g., a STY peptide), fusion proteins, or composition of the present invention, or one or more nucleic acids of the present invention in one or more sealed vials. The kits may include one or more components, such as vials, syringes, tubes, and instructions for use.

In some further embodiments there is provided a pharmaceutical composition of the invention comprised in a bottle where the bottle includes an exit portal that enables drop-wise administration of the composition. In some cases, a pharmaceutical composition comprised in a bottle comprises multiple doses. However, in certain aspects a bottle comprises a single dose unit for administration to one or two eyes; for example, a single dose unit may be comprised in 1-2 drops of the formulation. As used herein the term “bottle” refers to any fluid container such as an ampoule, dropper or syringe.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Gene structure of RTEF-1. The second STY domain is present within the 651 repressor isoform but absent from the 447 enhancer isoform. To test whether the STY domain alone can mediate repressor activity, the inventors synthesized STY linked to a cell penetrating peptide derived from tat (RMR). The STY domain is 26 amino acids in length and the RMR is 10 amino acids in length, making the STY-RMR peptide 36 amino acids long.

FIG. 2—RTEF is expressed in a variety of human tumor specimens.

FIG. 3—Proliferation of a breast cancer cell line incubated with STY-RMR for three days (XTT).

FIG. 4—Proliferation of a retinoblastoma cell line (Y 79) incubated with STY-RMR for three days (XTT).

FIG. 5—Proliferation of retinoblastoma cell line (Y 97) incubated with STY-RMR for three days (XTT).

FIGS. 6A-B—Proliferation of Mel270 incubated with STY-RMR for three days (XTT) (FIG. 6A). Proliferation of Mel202 incubated with STY-RMR for three days (XTT) (FIG. 6B).

FIGS. 7A-B—Proliferation of the ARPE-19 cell line incubated with STY-RMR for three days (XTT) (FIG. 7A). Proliferation of the RF-6A cell line incubated with STY-RMR for three days (XTT) (FIG. 7B).

FIG. 8—ELISA for VEGF inhibition by STY-RMR in Mel270 incubated for three days.

FIG. 9—ELISA for VEGF inhibition by STY-RMR in ARPE-19 incubated for three days.

FIG. 10—ELISA for VEGF inhibition by STY-RMR in ocular endothelial cells (RF/6A) incubated for three days.

FIG. 11—ELISA for VEGF inhibition by STY-RMR in breast cancer cells incubated for three days.

FIG. 12—Proliferation of breast cancer cells incubated with STY-RMR twice a day for three days (XTT).

### DESCRIPTION OF ILLUSTRATIVE EMBODIMENTS

In some aspects, peptides are provided that can inhibit tumor and/or endothelial cell proliferation. Alternative processing of RTEF-1 mRNA results in the production of different proteins that are able to either stimulate or inhibit VEGF gene transcription. The inventors tested short peptide fragments (e.g., STY-RMR) of an inhibitory RTEF-1 isoform for the ability to inhibit tumor and/or endothelial cell proliferation.

Using functional short peptide domains derived from the 651 RTEF-1 isoform may, in some embodiments, be used to treat ocular tumors and/or other VEGF-dependent neovascular diseases. Significant dose-dependent inhibition of cell proliferation was observed upon treatment with STY-RMR (SEQ ID NO:2). As shown in the below examples, maximal inhibition of ocular melanoma (Mel 202 and Mel 207) cell proliferation was observed at a dose of 30 mg/100 mL of STY-RMR (87% and 60% inhibition, respectively). At the same dose, more than 50% inhibition was observed in retinoblastoma and breast cancer cells (P<0.001). Significant inhibition of primate ocular endothelial cell proliferation (42% at 30 mg/100 mL (P<0.001) was seen, and retinal pigment epithelial cells showed a 75% inhibition (P=0.007). Secreted VEGF was decreased in the media of all tested cell lines that were exposed to STY-RMR. Inhibition of proliferation and VEGF production within ocular endothelial cells indicates that this agent may be used to treat age-related macular degeneration (ARMD) and/or diabetic retinopathy (DR).

### I. Polypeptides

The present invention concerns, in some aspects, polypeptides that include a RTEF-1 or STY amino acid sequence and a cell importation signal sequence. As used herein, a “polypeptide” generally is defined to refer to a peptide sequence of at least two amino acid residues. The term “amino acid” not only encompasses the 20 common amino acids in naturally synthesized proteins, but also includes any modified, unusual, or synthetic amino acids. One of ordinary skill in the art would be familiar with modified, unusual, or synthetic amino acids.

In some embodiments, some peptides or polypeptides provided herein are chimeric in that they comprise a RTEF-1 amino acid sequence and a cell importation signal sequence. The polypeptides set forth herein may comprise one or more cell importation signal, which may or may not be identical. Similarly, the polypeptides set forth herein may comprise one or more RTEF-1 amino acid sequence, which may or may not be identical.

In some embodiments, the polypeptide is a fusion polypeptide that includes a RTEF-1 or STY amino acid sequence linked at the N- or C-terminus to a cell importation signal. In some embodiments, the polypeptide comprises a linker interposed between the RTEF-1 or STY amino acid sequence and the cell importation signal.

A. Anti-Angiogenic Peptides

The STY peptide is a 26 amino acid peptide having the following sequence: SSFYGVSSQYESPENMIITCSTKVCS (SEQ ID NO:1). In some preferred embodiments, the STY peptide or a peptide containing the STY peptide motif is synthetically produced. In other embodiments, the STY peptide or a peptide containing the STY peptide motif may be recombinantly produced. In embodiments relating to a peptide (e.g., a synthetically produced peptide) comprising or consisting of the STY peptide sequence, the peptide may be 26, 27, 28, 29, 30, 31, 32, 33, 34, 35, 36, 37, 38, 39, 40, 41, 42, 43, 44, 45, 46, 47 or more amino acids in length. As used herein the phrase “STY peptide” refers to a peptide comprising or consisting of SEQ ID NO:1 that is shorter than an RTEF-1 polypeptide, optionally conjugated or fused to one or more peptide or protein (e.g., a cell importation signal, cell targeting moiety, linker, antibody, or antibody fragment, etc.). The STY peptide may have an amino acid sequence at least 95% identical or at least 97% identical to SEQ ID NO:1. Additionally, it is anticipated that the STY peptide may comprise 1, 2, or 3 mutations (e.g., conservative mutations, substitution mutations, or deletions) in SEQ ID NO:1 while retaining an ability, e.g., to inhibit tumor or endothelial cell proliferation.

B. Cell Importation Signals and Cell Targeting Moieties

1. Cell Importation Signals

A peptide of the present invention may comprise or be coupled to a cell importation peptide or a cellular internalization transporter (e.g., via a peptide bond, linker, or cleavable linker). As used herein the terms “cell penetrating peptide,” “cell importation peptide,” “cellular internalization transporter,” and “membrane translocation domain” are used interchangeably and refer to segments of polypeptide sequence that allow or promote a polypeptide to cross the cell membrane, such as the plasma membrane of a eukaryotic cell. Examples of cell importation signals include, but are not limited to, polyarginine sequences, segments derived from HIV Tat (e.g., GRKKRRQRRRPPQ, SEQ ID NO:23; or RKKRRQRRR, SEQ ID NO: 24), herpes virus VP22, the Drosophila Antennapedia homeobox gene product (RQPKIWFPNRRKPWKK; SEQ ID NO:25), protegrin I, Penetratin (RQIKIWFQNRRMKWKK; SEQ ID NO:26), Antp-3A (Antp mutant), Buforin II Transportan, MAP (model amphipathic peptide), K-FGF, Ku70, Prion, pVEC, Pep-I, SynB1, Pep-7, HN-1, KALA, R11, K11, or melittin (GIGAVLKVLTTGLPALISWIKRKRQQ; SEQ ID NO:27). In some embodiments, the cell importation signal is not a peptide; for example, the cell importation signal may be, in some embodiments, BGSC (Bis-Guanidinium-Spermidine-Cholesterol) or BGTC (BisGuanidinium-Tren-Cholesterol). Further cell importation sequences that may be used according to the embodiments include, without limitation, the T1 (TKIESLKEHG; SEQ ID NO:28), T2 (TQIENLKEKG; SEQ ID NO:29), (AALEALAEALEALAEALEALAEAAAA; SEQ ID NO:30), INF7 (GLFEAIEGFIENGWEGMIEGWYGCG; SEQ ID NO:31) CPP sequences or polyarginine sequences such as RMRRMRRMRR (SEQ ID NO:4); RGRRGRRGRR (SEQ ID NO:5); RRRRRRRRRR (SEQ ID NO:6); RARRARRARR (SEQ ID NO:7); RTRRTRRTRR (SEQ ID NO:8); RSRRSRRSRR (SEQ ID NO:9); RVRRVRRVRR (SEQ ID NO:10); RKRRKRRKRR (SEQ ID NO: ii); RRRRRRR (SEQ ID NO:12); RRRRRRRR (SEQ ID NO:13); RRRRRRRRR (SEQ ID NO:14); RRRRRRRRRRR (SEQ ID NO:15); RRRRRRRRRRRR (SEQ ID NO:16); RRRRRRRRRRRRR (SEQ ID NO:17); RRRRRRRRRRRRRR (SEQ ID NO:18); or RRRRRRRRRRRRRRR (SEQ ID NO:19). Poly-R sequences may vary in length, e.g., from 3, 4, 5, 6, 7, 8, 9, 10, 11, 12, 13, 14, or 15 R amino acids in length. The N-terminus of the cell importation signal sequence may be modified, for example, by adding a lipid moiety, myristolation, or acylation, e.g., to improve uptake and/or stability.

Cell importation signals for use herein may be covalently conjugated (e.g., chemically fused or attached, expressed as a fusion construct, etc.) with a STY peptide to promote transport of the STY peptide across a cell membrane. Cell importation signals that may be used include, e.g., peptides (e.g., cell penetration peptides), polypeptides, hormones, growth factors, cytokines, aptamers or avimers. Furthermore, a cell importation signal may mediate non-specific cell internalization or may be a cell targeting moiety that is internalized in a subpopulation of targeted cells.

Any cell importation signal sequence that can facilitate entry of a STY amino acid sequence into a cell is contemplated as a cell importation signal sequence of the present invention. In some embodiments, the cell importation signal sequence includes a motif of 3, 4, 5, 6, 7, 8, 9, 10, 11, 12, 13, 14, or 15 amino acids in length, and the cell importation signal sequence may include at least one arginine amino acid residue and at least one methionine amino acid residue. The cell importation signal may be synthetically or recombinantly produced. The arginine amino acid residue and the methionine amino acid residue may be consecutive residues within the motif, or they may be separated by one or more intervening amino acids. For example, the cell importation signal sequence may be the 10 amino acid RMR sequence: RMRRMRRMRR (SEQ ID NO:4). In some embodiments, the cell importation signal sequence includes more than one motif of two to fifteen amino acids, where each motif includes at least one arginine amino acid residue and at least one methionine amino acid residue. The motifs may include identical amino acid sequences or may have distinct amino acid sequences. Methionine/arginine-rich repeat motifs are discussed in Datar et al. (1993). Non-limiting examples of cell importation signal sequences are set forth in Table 1.

TABLE 1
Examples of Cell Importation Signal Sequences

| Sequence | SEQ ID NO: |
|---|---|
| RMRRMRRMRR | 4 |
| RGRRGRRGRR | 5 |
| RRRRRRRRRR | 6 |
| RARRARRARR | 7 |
| RTRRTRRTRR | 8 |
| RSRRSRRSRR | 9 |
| RVRRVRRVRR | 10 |
| RKRRKRRKRR | 11 |
| RRRRRRR | 12 |
| RRRRRRRR | 13 |
| RRRRRRRRR | 14 |
| RRRRRRRRRRR | 15 |
| RRRRRRRRRRRR | 16 |
| RRRRRRRRRRRRR | 17 |
| RRRRRRRRRRRRRR | 18 |
| RRRRRRRRRRRRRRR | 19 |
| RQPKIWFPNRRKPWKK | 25 |
| | (Antp) |
| RQIKIWFQNRRMKWKK | 26 |
| | (Penetratin) |
| RKKRRQRRR | 24 |
| | (Tat) |

The herein provided polypeptides may, in certain embodiments, be directly contacted to a tissue in a subject. However, efficiency of cytoplasmic localization of the provided polypeptide may be enhanced in some embodiments by a cellular internalization transporter chemically linked in cis or trans with the polypeptide. Efficiency of cell internalization transporters are enhanced further by light or co-transduction of cells with Tat-HA peptide.

Thus, the provided polypeptide can comprise a cellular internalization transporter or sequence. The cellular internalization sequence can be any internalization sequence known or newly discovered in the art, or conservative variants thereof. The cellular internalization peptide may comprise D-amino acids or be D-isomers of a peptide or amino acid sequence. In some embodiments the cellular internalization peptide comprises or consists of L-amino acids.

Thus, the provided polypeptide can further comprise amino acid sequences and other molecules described in, e.g., Bucci et al., 2000; Derossi et al., 1994; Fischer et al., 2000; Frankel and Pabo, 1988; Green and Loewenstein, 1988; Park et al., 2000; Pooga et al., 1998; Oehlke et al., 19989; Lin et al., 1995; Sawada et al., 2003; Lundberg et al., 2002; Morris et al., 2001; Rousselle et al., 2000; Gao et al., 2002; Hong and Clayman, 2000.

2. Cell Targeting Moieties

In some embodiments, a STY peptide may be expressed as a fusion protein or chemically attached to a cell targeting moiety to selectively target the construct containing the STY peptide to a particular subset of cells such as, e.g., cancerous cells, tumor cells, endothelial cells. For example, in some embodiments, the cell targeting moiety is an antibody. In general the term antibody includes, but is not limited to, polyclonal antibodies, monoclonal antibodies, single chain antibodies, humanized antibodies, minibodies, dibodies, tribodies as well as antibody fragments, such as Fab′, Fab, F(ab′)2, single domain antibodies and any mixture thereof. In some cases it is preferred that the cell targeting moiety is a single chain antibody (scFv). In a related embodiment, the cell targeting domain may be an avimer polypeptide. Therefore, in certain cases the cell targeting constructs of the invention are fusion proteins comprising a STY peptide and a scFv or an avimer. In some very specific embodiments the cell targeting construct is a fusion protein comprising a STY peptide fused to a single chain antibody.

In certain aspects of the invention, a cell targeting moiety may be a growth factor. For example, transforming growth factor, epidermal growth factor, insulin-like growth factor, fibroblast growth factor, B lymphocyte stimulator (BLyS), heregulin, platelet-derived growth factor, vascular endothelial growth factor (VEGF), or hypoxia inducible factor may be used as a cell targeting moiety according to the invention. These growth factors enable the targeting of constructs to cells that express the cognate growth factor receptors. For example, VEGF can be used to target cells that express FLK-1 and/or Flt-1. In still further aspects, the cell targeting moiety may be a polypeptide BLyS (e.g., see U.S. 2006/0171919).

In further aspects of the invention, a cell targeting moiety may be a hormone. Some examples of hormones for use in the invention include, but are not limited to, human chorionic gonadotropin, gonadotropin releasing hormone, an androgen, an estrogen, thyroid-stimulating hormone, follicle-stimulating hormone, luteinizing hormone, prolactin, growth hormone, adrenocorticotropic hormone, antidiuretic hormone, oxytocin, thyrotropin-releasing hormone, growth hormone releasing hormone, corticotropin-releasing hormone, somatostatin, dopamine, melatonin, thyroxine, calcitonin, parathyroid hormone, glucocorticoids, mineralocorticoids, adrenaline, noradrenaline, progesterone, insulin, glucagon, amylin, erythropoitin, calcitriol, calciferol, atrial-natriuretic peptide, gastrin, secretin, cholecystokinin, neuropeptide Y, ghrelin, PYY3-36, insulin-like growth factor-1, leptin, thrombopoietin, angiotensinogen, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, or IL-36. Targeting constructs that comprise a hormone may enable methods of targeting cell populations that comprise extracelluar receptors for the indicated hormone.

In yet further embodiments of the invention, cell targeting moieties may be cytokines. For example, IL1, IL2, IL3, IL4, IL5, IL6, IL7, IL8, IL9, IL10, IL11, IL12, IL13, IL14, IL15, IL-16, IL-17, IL-18, granulocyte-colony stimulating factor, macrophage-colony stimulating factor, granulocyte-macrophage colony stimulating factor, leukemia inhibitory factor, erythropoietin, granulocyte macrophage colony stimulating factor, oncostatin M, leukemia inhibitory factor, IFN-γ, IFN-α, IFN-β, LT-β, CD40 ligand, Fas ligand, CD27 ligand, CD30 ligand, 4-1BBL, TGF-β, IL 1α, IL-1β, IL-1 RA, MIF and IGIF may all be used as targeting moieties according to the invention.

In certain aspects, a cell targeting moiety of the invention may be a cancer cell-targeting moiety. It is well known that certain types of cancer cells aberrantly express surface molecules that are unique as compared to surrounding tissue. Thus, cell targeting moieties that bind to these surface molecules may enable the targeted delivery of STY peptides specifically to the cancers cells. For example, a cell targeting moiety may bind to and be internalized by a lung, breast, brain, prostate, spleen, pancreatic, cervical, ovarian, head and neck, esophageal, liver, skin, kidney, leukemia, bone, testicular, colon, or bladder cancer cell. The skilled artisan will understand that the effectiveness of a cancer cell-targeted STY peptide may, in some cases, be contingent upon the expression or expression level of a particular cancer marker on the cancer cell. Thus, in certain aspects, there are provided methods for treating a cancer with a targeted STY peptide comprising determining whether (or to what extent) the cancer cell expresses a particular cell surface marker and administering targeted STY peptide therapy (or another anticancer therapy) to the cancer cells depending on the expression level of a marker gene or polypeptide.

As discussed above, a cell targeting moiety according to the invention may be, for example, an antibody. For instance, a cell targeting moiety according the invention may bind to a skin cancer cell, such as a melanoma cell. It has been demonstrated that the gp240 antigen is expressed in a variety of melanomas but not in normal tissues. Thus, in certain aspects of the invention, there are provided cell targeting constructs comprising a STY peptide and a cell targeting moiety that binds to gp240. In some instances, the gp240 binding molecule may be an antibody, such as the ZME-018 (225.28S) antibody or the 9.2.27 antibody. In some embodiments, the gp240 binding molecule may be a single chain antibody, such as the scFvMEL antibody.

In yet further specific embodiments of the invention, cell targeting constructs may be directed to breast cancer cells. For example cell targeting moieties that bind to Her-2/neu, such as anti-Her-2/neu antibodies, may conjugated to a STY peptide. One example of such cell targeting constructs are fusion proteins comprising the single chain anti-Her-2/neu antibody scFv23 and a STY peptide. Other scFv antibodies, such as scFv(FRP5), that bind to Her-2/neu may also be used in the compositions and methods of the present invention (von Minckwitz et al., 2005).

In certain additional embodiments of the invention, it is envisioned that cancer cell-targeting moieties according to invention may have the ability to bind to multiple types of cancer cells. For example, the 8H9 monoclonal antibody and the single chain antibodies derived therefrom bind to a glycoprotein that is expressed on breast cancers, sarcomas and neuroblastomas (Onda et al., 2004). Another example is the cell targeting agents described in U.S. Appln. 2004/005647 and in Winthrop et al. (2003) that bind to MUC-1, an antigen that is expressed on a variety of cancer types. Thus, it will be understood that in certain embodiments, cell-targeting constructs according the invention may be targeted against a plurality of cancer or tumor types.

C. Linkers/Coupling Agents

In some embodiments, an RTEF-1 peptide or STY peptide of the present invention may be chemically attached to another group such as, e.g., a cell targeting moiety. If desired, the compound of interest may be joined via a biologically-releasable bond, such as a selectively-cleavable linker or amino acid sequence. For example, peptide linkers that include a cleavage site for an enzyme preferentially located or active within a tumor environment are contemplated. Exemplary forms of such peptide linkers are those that are cleaved by urokinase, plasmin, thrombin, Factor IXa, Factor Xa, or a metallaproteinase, such as collagenase, gelatinase, or stromelysin.

Additionally, while numerous types of disulfide-bond containing linkers are known which can successfully be employed to conjugate moieties, certain linkers will generally be preferred over other linkers, based on differing pharmacologic characteristics and capabilities. For example, linkers that contain a disulfide bond that is sterically “hindered” may be preferred, due to their greater stability in vivo, thus preventing release of the moiety prior to binding at the site of action.

Additionally, any other linking/coupling agents and/or mechanisms known to those of skill in the art can be attached to a peptide of the present invention, such as, for example, amide linkages, ester linkages, thioester linkages, ether linkages, thioether linkages, phosphoester linkages, phosphoramide linkages, anhydride linkages, disulfide linkages, ionic and hydrophobic interactions, or combinations thereof.

Cross-linking reagents are used to form molecular bridges that tie together functional groups of two different molecules, e.g., a stablizing and coagulating agent. However, it is contemplated that dimers or multimers of the same analog can be made or that heteromeric complexes comprised of different analogs can be created. To link two different compounds in a step-wise manner, hetero-bifunctional cross-linkers can be used that eliminate unwanted homopolymer formation. Examples of hetero-bifunctional cross linkers that may be used to attach an RTEF-1 peptide or Sty peptide of the present invention to, e.g., a cell importation signal are provided below in Table 2.

TABLE 2
HETERO-BIFUNCTIONAL CROSS-LINKERS

| | | | Spacer Arm |
|---|---|---|---|
| | | | Length\after |
| linker | Reactive Toward | Advantages and Applications | cross-linking |
| SMPT | Primary amines | Greater stability | 11.2 A |
| | Sulfhydryls | | |
| SPDP | Primary amines | Thiolation | 6.8 A |
| | Sulfhydryls | Cleavable cross-linking | |
| LC-SPDP | Primary amines | Extended spacer arm | 15.6 A |
| | Sulfhydryls | | |
| Sulfo-LC-SPDP | Primary amines | Extended spacer arm | 15.6 A |
| | Sulfhydryls | Water-soluble | |
| SMCC | Primary amines | Stable maleimide reactive group | 11.6 A |
| | Sulfhydryls | Enzyme-antibody conjugation | |
| | | Hapten-carrier protein conjugation | |
| Sulfo-SMCC | Primary amines | Stable maleimide reactive group | 11.6 A |
| | Sulfhydryls | Water-soluble | |
| | | Enzyme-antibody conjugation | |
| MBS | Primary amines | Enzyme-antibody conjugation | 9.9 A |
| | Sulfhydryls | Hapten-carrier protein conjugation | |
| Sulfo-MBS | Primary amines | Water-soluble | 9.9 A |
| | Sulfhydryls | | |
| SIAB | Primary amines | Enzyme-antibody conjugation | 10.6 A |
| | Sulfhydryls | | |
| Sulfo-SIAB | Primary amines | Water-soluble | 10.6 A |
| | Sulfhydryls | | |
| SMPB | Primary amines | Extended spacer arm | 14.5 A |
| | Sulfhydryls | Enzyme-antibody conjugation | |
| Sulfo-SMPB | Primary amines | Extended spacer arm | 14.5 A |
| | Sulfhydryls | Water-soluble | |
| EDC/Sulfo-NHS | Primary amines | Hapten-Carrier conjugation | 0 |
| | Carboxyl groups | | |
| ABH | Carbohydrates | Reacts with sugar groups | 11.9 A |
| | Nonselective | | |

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane. The use of such cross-linkers is well understood in the art. Another embodiment involves the use of flexible linkers.

D. Protein Purification

In some embodiments of the present invention, the peptide or polypeptide has been purified. Generally, “purified” will refer to a polypeptide composition that has been subjected to fractionation to remove various other components, and which composition substantially retains its expressed biological activity. Where the term “substantially purified” is used, this designation will refer to a composition in which the polypeptide or peptide forms the major component of the composition, such as constituting about 50% to about 99.9% or more of the proteins in the composition.

Various methods for quantifying the degree of purification of the polypeptide will be known to those of skill in the art in light of the present disclosure. Exemplary techniques include high performance liquid chromatography, ion exchange chromatography, gel electrophoresis, affinity chromatography and the like. The actual conditions used to purify a particular polypeptide will depend, in part, on factors such as net charge, hydrophobicity, hydrophilicity, etc., and will be apparent to those having skill in the art.

### II. Nucleic Acid Delivery

A. Viral Vectors

The ability of certain viruses to infect cells or enter cells via receptor mediated endocytosis, and to integrate into the host cell genome and express viral genes stably and efficiently have made them attractive candidates for the transfer of foreign nucleic acids into cells (e.g., mammalian cells). A nucleic acid that encodes a STY peptide or a peptide comprising a STY motif of the present invention may be incorporated into a viral vector. Non-limiting examples of viral vectors that may be used to deliver a nucleic acid of the present invention are described below.

1. Adenoviral Vectors

A particular method for delivery of nucleic acid involves the use of an adenovirus expression vector. Although adenoviral vectors are known to have a low capacity for integration into genomic DNA, this feature is counterbalanced by the high efficiency of gene transfer afforded by these vectors. “Adenoviral expression vector” is meant to include those constructs containing adenoviral sequences sufficient to (a) support packaging of the construct and (b) to ultimately express in a tissue or cell the specific construct that has been cloned therein. Knowledge of the genetic organization of adenovirus, a 36 kb, linear, double-stranded DNA virus, allows substitution of large pieces of adenoviral DNA with foreign sequences up to 7 kb (Grunhaus and Horwitz, 1992).

2. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno associated virus (AAV) is an attractive vector system for use in the delivery of STY expression cassettes of the present invention as it has a high frequency of integration and can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

3. Retroviral Vectors

Retroviruses have promise as delivery vectors in therapeutics due to their ability to be packaged in special cell lines, infect a broad spectrum of species and cell types, and integrate their genes into the host genome, transferring a large amount of foreign genetic material (Miller, 1992).

In order to construct a retroviral vector, a nucleic acid (e.g., one encoding a STY peptide) is inserted into the viral genome in the place of certain viral sequences to produce a virus that is replication defective. In order to produce virions, a packaging cell line containing the gag, pol, and env genes, but without the LTR and packaging components, is constructed (Mann et. al., 1983). When a recombinant plasmid containing a cDNA, together with the retroviral LTR and packaging sequences, is introduced into a special cell line (e.g., by calcium phosphate precipitation, for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Methods for delivery of antiangiogenic molecules with lentiviral vectors have been previously described, see, for example, U.S. Pat. No. 7,122,181, U.S. Patent App. Publ. Nos. 2009/0148936, 2006/0062765, 2003/0082159, and 2002/0114783, each of which is incorporated herein by reference in its entirety. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentiviruses include the Human Immunodeficiency Viruses, HIV-1 and HIV-2, and the Simian Immunodeficiency Virus, SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus is capable of infecting a non-dividing cell wherein a suitable host cell is transfected with two or more vectors carrying the packaging functions, namely gag, pol and env, as well as rev and tat, as described in U.S. Pat. No. 5,994,136, incorporated herein by reference. One may target the recombinant virus by linkage of the envelope protein with an antibody or a particular ligand for targeting to a receptor of a particular cell type. By inserting a sequence (including a regulatory region) of interest into the viral vector, along with another gene that encodes the ligand for a receptor on a specific target cell, for example, the vector is now target-specific.

4. Other Viral Vectors

Other viral vectors may be employed as vaccine constructs in embodiments of the present invention. Vectors derived from viruses, such as vaccinia virus (Ridgeway, 1988; Baichwal and Sugden, 1986; Coupar et al., 1988), sindbis virus, cytomegalovirus, and herpes simplex virus, may be employed. They offer several attractive features for various mammalian cells (Friedmann, 1989; Ridgeway, 1988; Baichwal and Sugden, 1986; Coupar et al., 1988; Horwich et al., 1990).

5. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. Another approach designed to allow specific targeting of retroviral vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

B. Vector Delivery and Cell Transformation

Suitable methods for nucleic acid delivery for transformation of an organelle, a cell, a tissue or an organism for use with the present invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA, such as by ex vivo transfection (Wilson et al., 1989; Nabel et al., 1989); by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

### III. Therapeutic Methods

A. Pharmaceutical Preparations

Therapeutic compositions for use in methods of the invention may be formulated into a pharmacologically acceptable format. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one STY peptide or nucleic acid active ingredient will be known to those of skill in the art in light of the present disclosure, as exemplified by Remington: The Science and Practice of Pharmacy, 21^{st} Ed. Lippincott Williams & Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington: The Science and Practice of Pharmacy, 21^{st} Ed. Lippincott Williams & Wilkins, 2005, incorporated herein by reference). A pharmaceutically acceptable carrier is preferably formulated for administration to a human, although in certain embodiments it may be desirable to use a pharmaceutically acceptable carrier that is formulated for administration to a non-human animal, such as a canine, but which would not be acceptable (e.g., due to governmental regulations) for administration to a human. Except insofar as any conventional carrier is incompatible with the active ingredient, its use in the therapeutic or pharmaceutical compositions is contemplated.

The actual dosage amount of a composition of the present invention administered to a subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 mg/kg/body weight, etc., can be administered, based on the numbers described above.

In particular embodiments, the compositions of the present invention are suitable for application to mammalian eyes. For example, the formulation may be a solution, a suspension, or a gel. In some embodiments, the composition is administered via a bioerodible implant, such as an intravitreal implant or an ocular insert, such as an ocular insert designed for placement against a conjunctival surface. In some embodiments, the therapeutic agent coats a medical device or implantable device.

In some embodiments, the formulation of the invention is applied to the eye in an aqueous solution in the form of drops (e.g., saline eye drops). These drops may be delivered from a single dose ampoule, which may preferably be sterile and thus rendering bacteriostatic components of the formulation unnecessary. Alternatively, the drops may be delivered from a multi-dose bottle, which may preferably comprise a device that extracts preservative from the formulation as it is delivered, such devices being known in the art. In some embodiments a therapeutic composition of the present invention (e.g., containing a STY peptide or a peptide comprising a STY peptide) may be administered via intravitreal injection.

In other aspects, components of the invention may be delivered to the eye as a concentrated gel or similar vehicle that forms dissolvable inserts that are placed beneath the eyelids.

Furthermore, the therapeutic compositions of the present invention may be administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil, and injectable organic esters, such as ethyloleate. Aqueous carriers include water, alcoholic/aqueous solutions, saline solutions, and parenteral vehicles, such as sodium chloride, Ringer's dextrose, etc. Intravenous vehicles include fluid and nutrient replenishers. Preservatives include antimicrobial agents, anti-oxidants, chelating agents, and inert gases. The pH and exact concentration of the various components the pharmaceutical composition are adjusted according to well known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve, or spray.

An effective amount of the therapeutic composition is determined based on the intended goal. The term “unit dose” or “dosage” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the protection desired. Thus, in some case dosages can be determined by measuring for example changes in serum insulin or glucose levels of a subject.

Precise amounts of the therapeutic composition may also depend on the judgment of the practitioner and are peculiar to each individual. Factors affecting the dose include the physical and clinical state of the patient, the route of administration, the intended goal of treatment (e.g., alleviation of symptoms versus attaining a particular serum insulin or glucose concentration) and the potency, stability, and toxicity of the particular therapeutic substance.

B. Additional Therapies

As discussed supra, in certain aspects, therapeutic methods of the invention may be used in combination or in conjunction with additional antiangiogenic or anticancer therapies.

1. Chemotherapy

In certain embodiments of the invention a STY peptide may be administered in conjunction with a chemo therapeutic agent. For example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, gemcitabien, navelbine, farnesyl-protein tansferase inhibitors, transplatinum, 5-fluorouracil, vincristin, Velcade, vinblastin and methotrexate, or any analog or derivative variant of the foregoing may used in methods according to the invention.

2. Radiotherapy

In certain further embodiments of the invention, compositions of the invention may be used to sensitize a cell to radiation therapy. Radiotherapy may include, for example, γ-rays, X-rays, and/or the directed delivery of radioisotopes to tumor cells. In certain instances microwaves and/or UV-irradiation may also used according to methods of the invention. Dosage ranges for X-rays range from daily doses of 50 to 200 roentgens for prolonged periods of time (e.g., about 3 to 4 weeks), to single doses of 2000 to 6510 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

3. Immunotherapy

Immunotherapeutics typically rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (e.g., a chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy may be used as part of a combined therapy, e.g., in conjunction with a gene therapy or administration of a STY peptide of the present invention. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B, Her-2/neu, gp240, and p155.

4. Gene Therapy

In yet another embodiment, a gene therapy may be administered to a subject such as a human patient before, after, or at the same time as a therapeutic cell targeting construct or STY peptide of the present invention. Delivery of a STY peptide in conjunction with a vector encoding one or more additional gene products may have a combined anti-hyperproliferative effect on target tissues. A variety of genes are encompassed within the invention, for example, a gene encoding p53 may be delivered in conjunction with STY peptide compositions.

5. Surgery

Approximately 60% of persons with cancer will undergo surgery of some type such as, e.g., a preventative, diagnostic, staging, curative, or palliative surgery. A curative surgery is a cancer treatment that may be used in conjunction with other therapies, such as a treatment of the present invention, chemotherapy, radiotherapy, hormonal therapy, gene therapy, immunotherapy and/or alternative therapies. A STY peptide therapy or gene therapy of the invention may be employed alone or in combination with a cytotoxic therapy as neoadjuvant surgical therapy (e.g., as to reduce tumor size prior to resection) or a therapy of the present invention may be administered as a postadjuvant surgical therapy, for example to sterilize a surgical bed following removal of part or all of a tumor.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of or all of the cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

6. Other Agents

Hormonal therapy may also be used in conjunction with the present invention or in combination with any other cancer therapy previously described. The use of hormones may be employed in the treatment of certain cancers, such as breast, prostate, ovarian, or cervical cancer, to lower the level or block the effects of certain hormones, such as testosterone or estrogen. This treatment is often used in combination with at least one other cancer therapy as a treatment option or to reduce the risk of metastases.

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

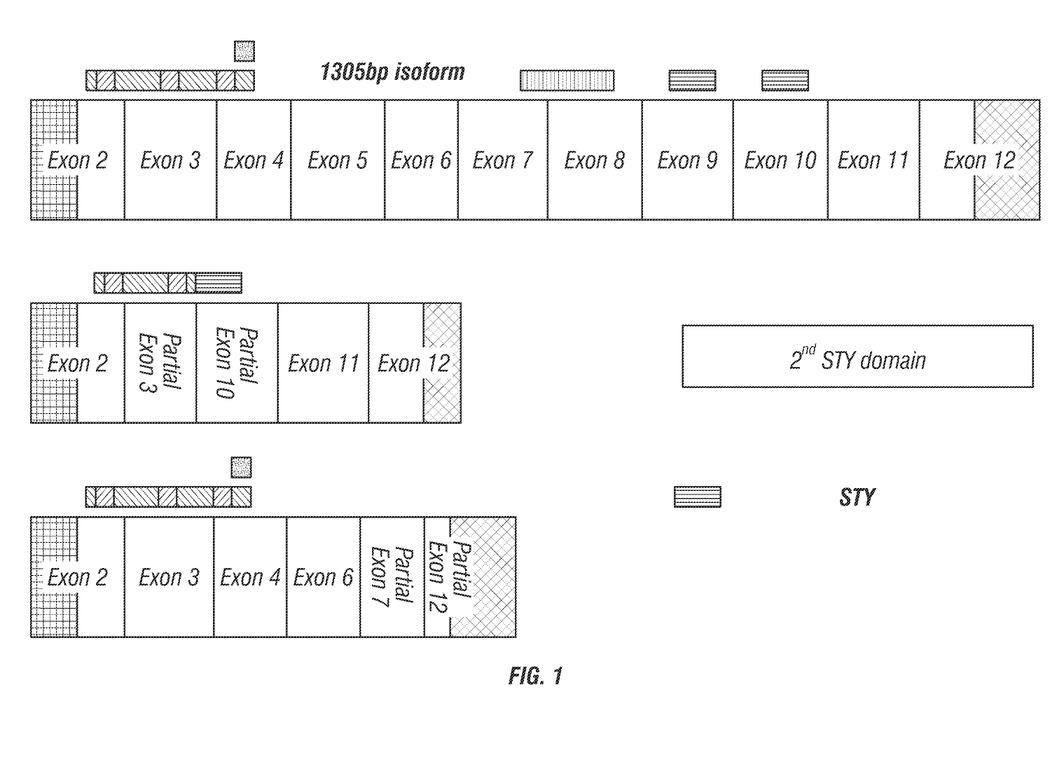

A 26 amino acid sequence corresponding to a Ser-Thr-Tyr domain within RTEF-1 (FIG. 1), linked to a 10 amino acid cell importation signal (RMR) was synthesized (GenScript NJ). Human ocular melanoma cells (Mel 270, Mel 202), retinoblastoma cells (Y79; ATTC, MD), primate ocular endothelial retina/choroid ocular endothelial cells (RF/6A; ATCC, MD), human retinal pigment epithelial cells (ARPE19; ATCC, MD), and the CRL 1500 breast cancer cell line (ATCC, MD) were plated into 96 well plates and cultured for 24 h. Recombinant STY-RMR peptide was added to the cell culture media at various concentrations (10 to 30 mg/100 mL).

Cell proliferation was assessed at 72 h using a colorimetric XTT assay (Roche Diagnostics, Indianpolis, Conn., USA). Cell proliferation was expressed as a percentage and compared with untreated control cell growth (N=3).

The amount of VEGF within the media was determined by VEGF ELISA (R&D Systems, MN, USA) and compared between STY-RMR treated and controls (N=3).

### Example 2

### Inhibition of Ocular Tumor Cell Growth with a RTEF-1 Peptide Fragment

To test whether the STY domain alone (which is present within the 651 repressor isoform but absent from the 447 enhancer isoform) can mediate repressor activity, the inventors synthesized STY linked to a cell importation signal derived from tat (RMR) (GenScript NJ) (FIG. 1).

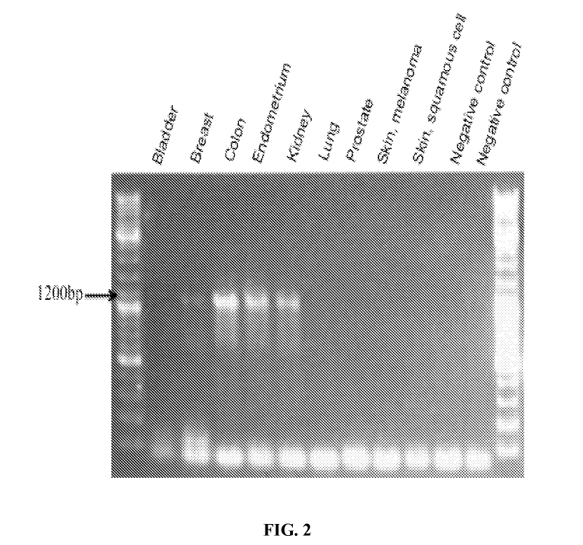

Related transcription enhancer factor 1 (RTEF-1) is a member of the TED DNA binding domain family, and it is present within ocular vascular endothelial cells and plays a role in the control of VEGF expression. The inventors have demonstrated that a variety of human tumors expressed isoforms of RTEF-1 (FIG. 2).

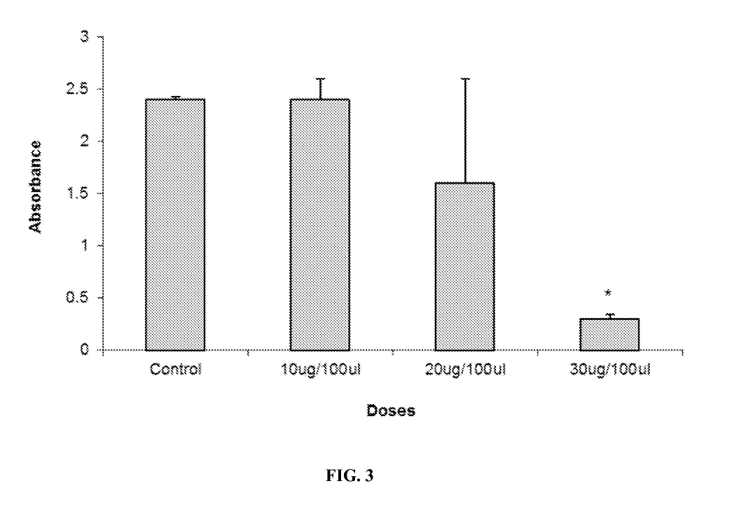

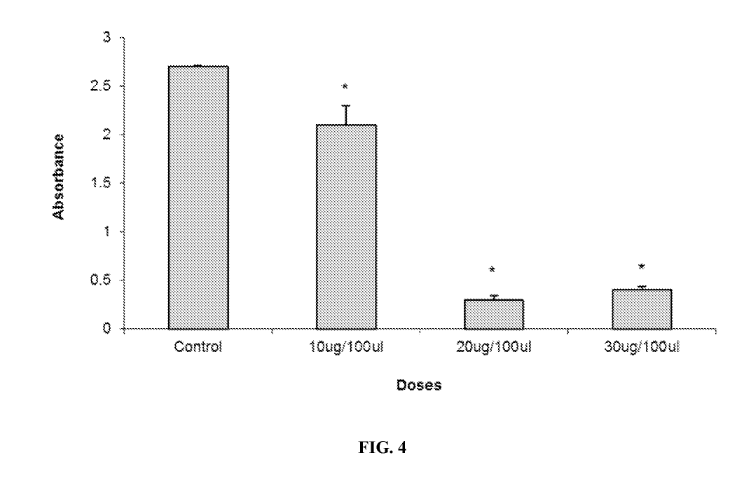

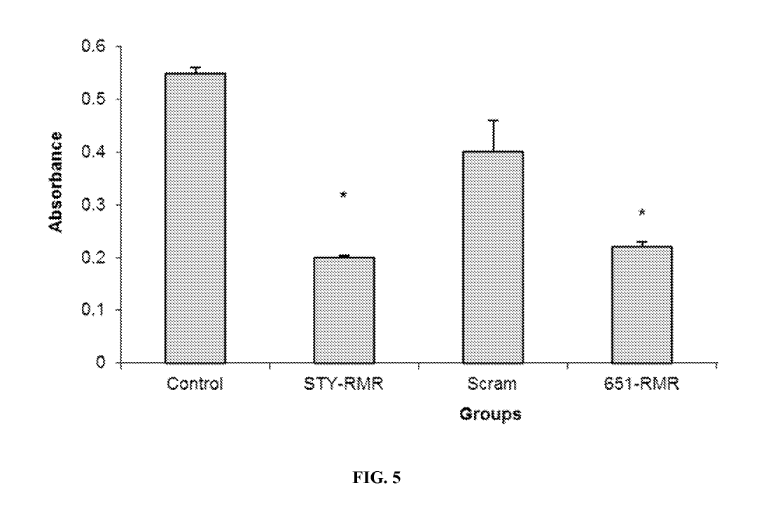

Breast cancer cells (CRL 1500) showed a very significant inhibition of 87% (P=0.000002) at (30 μg/100 μL) compared with untreated controls; the mean results of three independent experiments are shown (±SEM) in FIG. 3. The proliferation of retinoblastoma cells was also inhibited by STY-RMR peptide in a dose-dependent manner. The inventors observed 85% inhibition at 30 mg/100 mL, which is statistically different from control experiments in the absence of STY-RMR (P=0.00003). Mean results of three independent experiments are shown (±SEM) in FIG. 4.

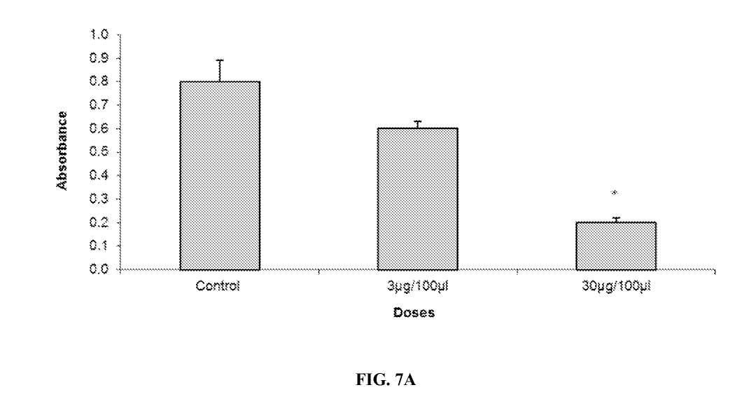

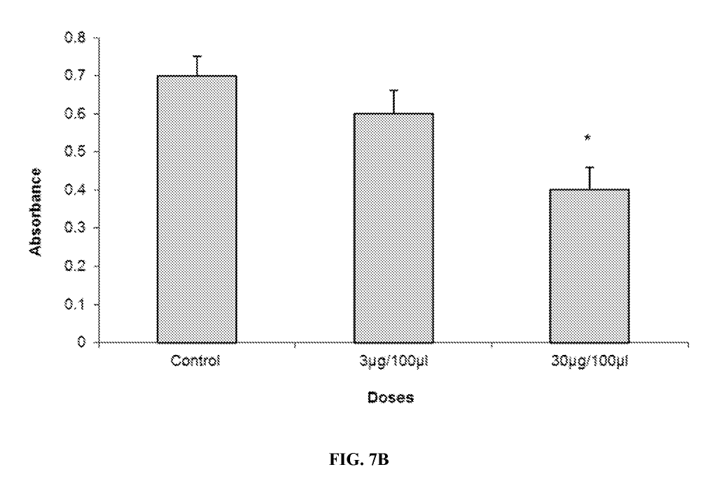

STY-RMR peptide can inhibit proliferation of ocular vascular endothelial (RF/6A) and retinal pigment epithelial cells (ARPE-19) (FIG. 7A). A (42%, P=0.001) inhibition of RF-6A (FIG. 7B) and 75% inhibition of ARPE-19 cells was observed with 30 mg/100 mL of STY-RMR (P=0.007). A dose-dependent response was also observed as inhibition of cell proliferation was obtained with 3 μg/100 μl of treatment.

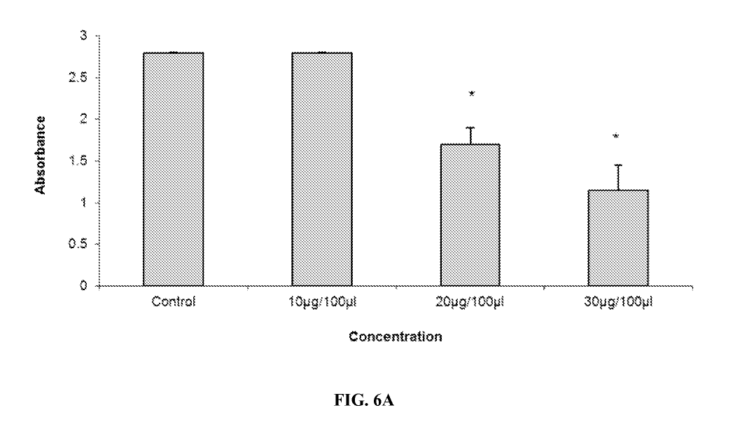

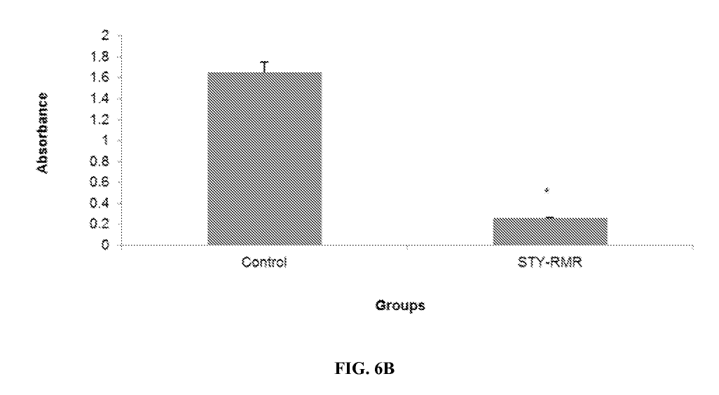

STY-RMR can inhibit cell proliferation in two different ocular melanoma cell lines. 87% inhibition was observed at 30 mg/100 mL for the Mel 202 cells. Mean results of three independent experiments are shown (±SEM) (P=0.001) in FIG. 6B. Significant inhibition (P=0.006) of 60% below control was also observed in Mel270 cells incubated at the same dose, and inhibition was dose-dependent (FIG. 6A).

A scrambled version was tested as a control and did not show any significant inhibition in retinoblastoma (Y 79) cells (FIG. 5).

The inventors further tested the potential of this agent to inhibit tumor cell lines at a lower range of daily doses and focused on breast cancer to optimize conditions of delivery and efficacy.

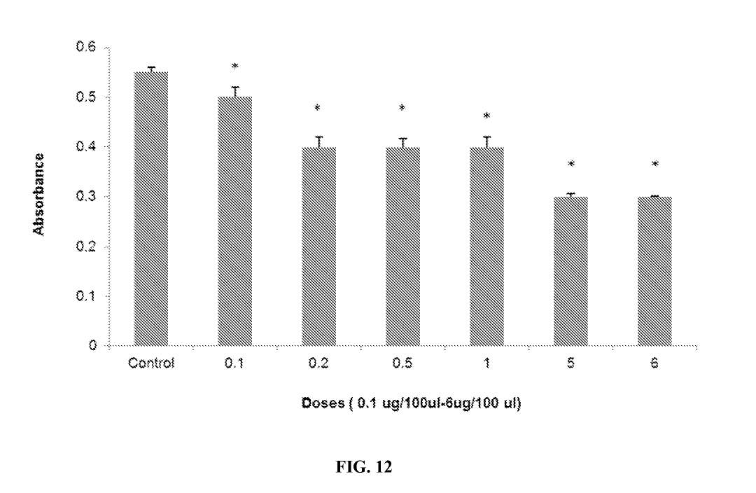

A significant inhibitory effect could be achieved by much lower daily doses twice a day for three days (FIG. 12), since breast cancer cell (CRL 1500) had 9% inhibition at 0.1 μg/100 μL (P=0.02), and 27% inhibition at 0.2, 0.5, and 1 μg/100 μL (P=0.02, 0.017, 0.02, respectively), however this inhibition was increased to 50% at (5 and 6 μg/100 μL) with much higher significance (P=0.00002, 0.0005), indicating that this factor may have similar metronomic effect observed for chemotherapeutic agents that can inhibit tumors either by high one shot or daily small doses due to thrompospondin release, which is known to be an endogenous anti-angiogenic factor. However, it cannot be assumed that the inhibitory effect of STY-RMR given once is cytotoxic at the highest dose despite the observation of a potent cell death induction at (30 μg/100 μL) since a significant VEGF reduction at the previous dose was also observed.

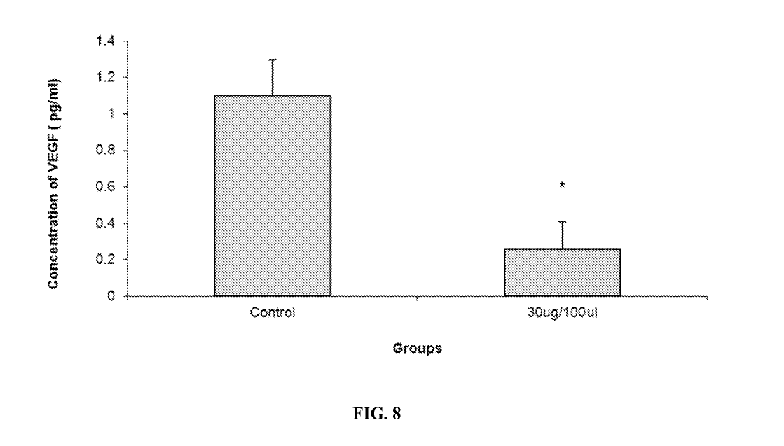

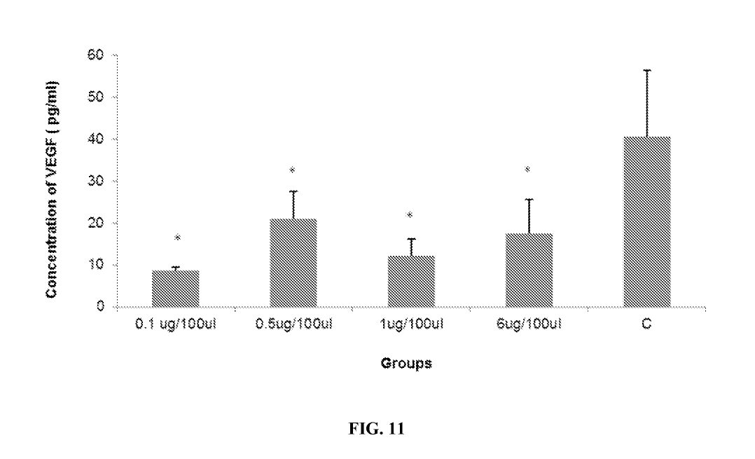

This inhibition in breast tumor cells was also associated with decreased VEGF levels by STY-RMR 78% (P=0.02) even at a low concentration (0.1 μg/100 μL) (FIG. 11). ELISA indicated that STY-RMR treatment is able to decrease secreted VEGF levels in ocular melanoma cell line 270 by STY-RMR peptide. The inventors observed 81% reduction in VEGF levels (P=0.01) at 30 mg/100 mL treatment (FIG. 8).

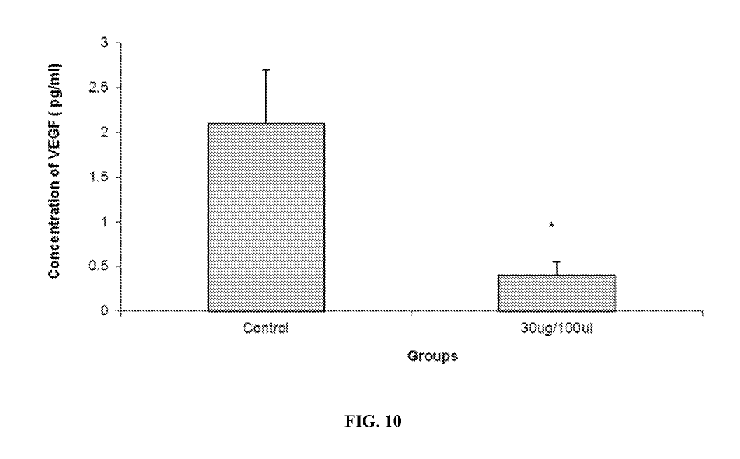

Levels of secreted VEGF is lower in ocular retinal-choroidal derived vascular endothelial cells after treatment with STY-RMR peptide. The inventors observed 80% at 30 mg/100 mL treatment (P=0.03) (FIG. 10).

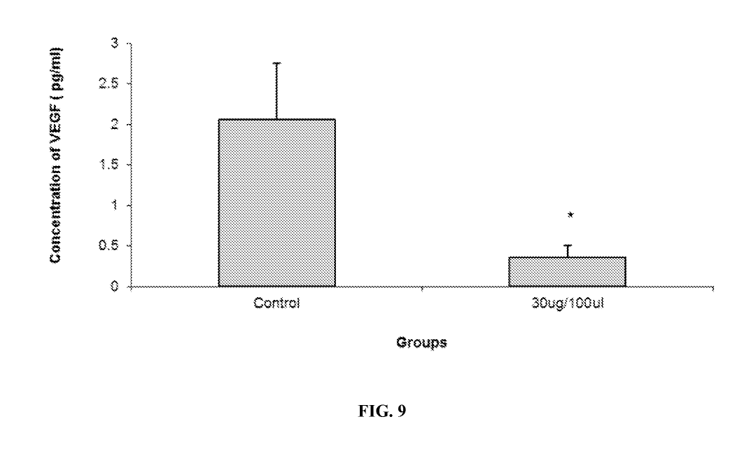

VEGF levels are inhibited in ARPE19 cells by STY-RMR peptide. An 88% reduction compared to control was observed after treatment with 30 mg/100 mL (P=0.05) (FIG. 9).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,684,611
U.S. Pat. No. 4,797,368
U.S. Pat. No. 4,952,500
U.S. Pat. No. 5,139,941
U.S. Pat. No. 5,302,523
U.S. Pat. No. 5,322,783
U.S. Pat. No. 5,384,253
U.S. Pat. No. 5,464,765
U.S. Pat. No. 5,538,877
U.S. Pat. No. 5,538,880
U.S. Pat. No. 5,550,318
U.S. Pat. No. 5,563,055
U.S. Pat. No. 5,580,859
U.S. Pat. No. 5,589,466
U.S. Pat. No. 5,591,616
U.S. Pat. No. 5,610,042
U.S. Pat. No. 5,656,610
U.S. Pat. No. 5,702,932
U.S. Pat. No. 5,736,524
U.S. Pat. No. 5,780,448
U.S. Pat. No. 5,789,215
U.S. Pat. No. 5,945,100
U.S. Pat. No. 5,981,274
U.S. Pat. No. 5,994,136
U.S. Pat. No. 5,994,624
U.S. Pat. No. 6,013,516
U.S. Pat. No. 7,122,181
U.S. Patent Appln. Publ. No. 2002/0114783
U.S. Patent Appln. Publ. No. 2003/0082159
U.S. Patent Appln. Publ. No. 2004/0005647
U.S. Patent Appln. Publ. No. 2006/0062765
U.S. Patent Appln. Publ. No. 2009/0117119
U.S. Patent Appln. Publ. No. 2006/0171919
U.S. Patent Appln. Publ. No. 2009/0148936
U.S. Patent Appln. Publ. No. 2012/0063994
PCT Appln. No. WO 94/09699
PCT Appln. No. WO 95/06128
An et al., Endothelial cells require related transcription enhancer factor-1 for cell-cell connections through the induction of gap junction proteins, Arterioscler. Thromb. Vasc. Biol., 32(8):1951-1959, 2012.
Appukuttan et al., Invest. Ophthalmol. Vis. Sci., 48(8):3775-3782, 2007.
Areán et al., Expression of vascular endothelial growth factor in retinoblastoma, Arch. Ophthalmol., 128(2):223-229, 2010.
Baichwal and Sugden, In: Gene Transfer, Kucherlapati (Ed.), Plenum Press, NY, 117-148, 1986.
Bareschino et al., The role of antiangiogenetic agents in the treatment of breast cancer, Curr. Med. Chem., 18(33):5022-5032, 2011.
Blomer et al., J. Virol., 71(9):6641-6649, 1997.
Bucci et al., Nat. Med., 6:1362-1367, 2000.
Cavazzoni et al., Malignant ascites: pathophysiology and treatment, Int. J. Clin. Oncol., 18:1-9, 2013.
Chen and Okayama, Mol. Cell Biol., 7(8):2745-2752, 1987.
Claesson and Spjuth, On mechanisms of reactive metabolite formation from drugs, Mini Rev. Med. Chem., 13(5):720-729, 2013.
Cotten et al., Proc. Natl. Acad. Sci. USA, 89(13):6094-6098, 1992.
Coupar et al., Gene, 68:1-10, 1988.
Crosby et al., Serum vascular endothelial growth factor (VEGF) levels correlate with number and location of micrometastases in a murine model of uveal melanoma, Br. J. Ophthalmol., 95(1):112-117, 2011.
Curiel, Nat. Immun., 13(2-3):141-164, 1994.
Datar et al., Nucleic Acids Res., 21(3):439-446, 1993.
Derossi et al., Biol. Chem., 269:10444-10450, 1994.
Fechheimer et al., Proc. Natl. Acad. Sci. USA, 84:8463-8467, 1987.
Ferrara, VEGF as a therapeutic target in cancer, Oncology, 69(Supp. 3):11-16, 2005.
Fischer et al, J. Pept. Res., 55:163-172, 2000.
Folkman, Angiogenesis: an organizing principle for drug discovery? Nat. Rev. Drug Discov., 6(4):273-286, 2007.
Fraley et al., Proc. Natl. Acad. Sci. USA, 76:3348-3352, 1979.
Frankel and Pabo, Cell, 55:1189-1193, 1988.
Friedmann, Science, 244:1275-1281, 1989.
Gao et al., Bioorg. Med. Chem., 10:4057-4065, 2002.
Gopal, Mol. Cell Biol., 5:1188-1190, 1985.
Graham and Van Der Eb, Virology, 52:456-467, 1973.
Green and Loewenstein, Cell, 55:1179-1188, 1988.
Grunhaus and Horwitz, Seminar in Virology, 3:237-252, 1992.
Harland and Weintraub, J. Cell Biol., 101(3):1094-1099, 1985.
Hong and Clayman, Cancer Res., 60:6551-6556, 2000.
Horwich et al. J. ViroL, 64:642-650, 1990.
Kaeppler et al., Plant Cell Reports, 9:415-418, 1990.
Kaneda et al., Science, 243:375-378, 1989.
Kato et al., J. Biol. Chem., 266:3361-3364, 1991.
Kelleher and Vos, Biotechniques, 17(6):1110-7, 1994.
Lee et al., Linking antibody Fc domain to endostatin significantly improves endostatin half-life and efficacy, Clin. Cancer Res., 14(5):1487-1493, 2008.
Laughlin et al., J. ViroL, 60(2):515-524, 1986.
Lebkowski et al., Mol. Cell. Biol., 8(10):3988-3996, 1988.
Lin et al, J. Biol Chem., 270:14255-14258, 1995.
Lundberg et al., Biochem. Biophys. Res. Comm., 299, 2002.
Maae et al., Prognostic impact of placenta growth factor and vascular endothelial growth factor a in patients with breast cancer, Breast Cancer Res. Treat., 133(1):257-265, 2012.
Maeda et al., TEF-1 transcription factors regulate activity of the mouse mammary tumor virus LTR. Biochem. Biophys. Res. Commun., 296(5):1279-1285, 2002.
Maloney et al., Induction of thrombospondin-1 partially mediates the anti-angiogenic activity of dexrazoxane, Br. J. Cancer, 101(6):957-966, 2009.
Mann et al., Cell, 33:153-159, 1983.
Matsuki et al., Anti-angiogenesis effect of 3′-sulfoquinovosyl-1′-monoacylglycerol via upregulation of thrombospondin 1, Cancer Sci., 103(8): 1546-1552, 2012.
McLaughlin et al., J. ViroL, 62(6):1963-1973, 1988.
Metcalfe et al., Decreased affinity of blood for oxygen in patients with low-output heart failure, Circ. Res., 25(1):47-51, 1969.
Miller et al, Am. J. Clin. Oncol., 15(3):216-221, 1992.
Morris et al., Nat. Biotechnol., 19:1173-1176, 2001.
Muzyczka, Curr. Topics Microbiol. Immunol., 158:97-129, 1992.
Nabel et al., Science, 244(4910): 1342-1344, 1989.
Naldini et al., Science, 272(5259):263-267, 1996.
Nicolas and Rubinstein, In: Vectors: A survey of molecular cloning vectors and their uses,
Rodriguez and Denhardt, eds., Stoneham: Butterworth, pp. 494-513, 1988.
Nicolau and Sene, Biochim. Biophys. Acta, 721:185-190, 1982.
Nicolau et al., Methods Enzymol., 149:157-176, 1987.
Oehlke et al., Biochim. Biophys. Acta., 1414; 127-139, 1998.
Omirulleh et al., Plant Mol. Biol., 21(3):415-428, 1993.
Onda et al., Cancer Res., 64:1419-1424, 2004.
Ooyama et al., Anti-angiogenic effect of 5-Fluorouracil-based drugs against human colon cancer xenografts, Cancer Lett., 267(1):26-36, 2008.
Park et al., Proc. Natl. Acad. Sci. USA, 97:8245-8250, 2000.
Paskind et al., Virology, 67:242-248, 1975.
Pastuskovas et al., Effects of anti-VEGF on pharmacokinetics, biodistribution, and tumor penetration of trastuzumab in a preclinical breast cancer model, Mol. Cancer Ther., 11(3):752-762, 2012.
Pooga et al, FASEB J., 12, 67-77, 1998.
Potrykus et al., Mol. Gen. Genet., 199(2):169-177, 1985.
Potter et al., Proc. Natl. Acad. Sci. USA, 81:7161-7165, 1984.
Ramaekers et al., Hypoxia disrupts the Fanconi anemia pathway and sensitizes cells to chemotherapy through regulation of UBE2T, Radiother. Oncol., 101(1):190-197, 2011.
Remington's Pharmaceutical Sciences, 18th Ed. Mack Printing Company, pp. 1289-1329, 1990.
Ridgeway, In: Vectors: A Survey of Molecular Cloning Vectors and Their Uses, Rodriguez et al. (Eds.), Stoneham: Butterworth, 467-492, 1988.
Rippe et al., Mol. Cell Biol., 10:689-695, 1990.
Rousselle et al., Mol. Pharmacol., 57:679-686, 2000.
Roux et al., Proc. Natl. Acad. Sci. USA, 86:9079-9083, 1989.
Sahin et al., Expression of vascular endothelial growth factor-A, matrix metalloproteinase-9, and extravascular matrix patterns and their correlations with clinicopathologic parameters in posterior uveal melanomas, Jpn. J. Ophthalmol., 51(5):325-331, 2007.
Sawada et al., Nature Cell Biol., 5:352-357, 2003.
Temin, In: Gene Transfer, Kucherlapati (Ed.), NY, Plenum Press, 149-188, 1986.
Tjin Tham Sjin et al., A 27-amino-acid synthetic peptide corresponding to the NH2-terminal zinc-binding domain of endostatin is responsible for its antitumor activity, Cancer Res., 65(9):3656-3663, 2005.
Toustrup et al., Hypoxia gene expression signatures as prognostic and predictive markers in head and neck radiotherapy, Semin. Radiat. Oncol., 22(2):119-127, 2012.
Tratschin et al., Mol. Cell. Biol., 4:2072-2081, 1984.
Tur-Kaspa et al., Mol. Cell Biol., 6:716-718, 1986.
von Minckwitz et al., Breast Cancer Res., 7:R616-626, 2005.
Wilson et al., Science, 244:1344-1346, 1989.
Winthrop et al., Clin. Cancer Res., 9:3845s-3853s, 2003.
Wong et al, Gene, 10:87-94, 1980.
Wu and Wu, Biochemistry, 27: 887-892, 1988.
Wu and Wu, J. Biol. Chem., 262:4429-4432, 1987.
Xia et al., Recent advances in hypoxia-inducible factor (HIF)-1 inhibitors, Eur. J. Med. Chem., 49:24-40, 2012.
Zhang et al., Hypoxia-induced expression of RTEF-1 (related transcriptional enhancer factor-1) in endothelial cells is independent of HIF-1 (hypoxia-inducible factor-1), Biochem. Biophys. Res. Commun., 381(3):333-338, 2009.
Zufferey et al., Nat. Biotechnol., 15(9):871-875, 1997.

What is claimed is:

1. A peptide comprising a region having an amino acid sequence at least 90% identical to SEQ ID NO:1, wherein the peptide does not comprise a full-length RTEF-1 polypeptide, an RTEF 669 isoform (SEQ ID NO:20), an RTEF 651 isoform (SEQ ID NO:21), or an RTEF 366 isoform (SEQ ID NO:22); and wherein the peptide can reduce VEGF promoter activity.

2. The peptide of claim 1, wherein the peptide comprises less than 121 contiguous amino acids of an RTEF-1 polypeptide.

3. The peptide of any one of claims 1-2, wherein the peptide is less than 121 amino acids in length.

4. The peptide of any one of claims 1-3, wherein the peptide comprises no more than 45 contiguous amino acids of an RTEF-1 polypeptide.

5. The peptide of any one of claims 1-4, wherein the peptide is less than 45 amino acids in length.

6. The peptide of any one of claims 1-5, wherein the peptide comprises an amino acid sequence at least 95% identical to SEQ ID NO: 1.

7. The peptide of claim 6, wherein the peptide comprises an amino acid sequence at least 97% identical to SEQ ID NO:1.

8. The peptide of claim 7, wherein the peptide comprises the sequence of SEQ ID NO:1.

9. The peptide of any one of claims 1-8, wherein the peptide is conjugated or fused to a cell importation signal sequence.

10. The peptide of any one of claims 1-8, wherein the peptide is covalently coupled to a cell importation signal sequence.

11. The peptide of any one of claims 9-10, wherein the cell importation signal sequence is the sequence of any one of SEQ ID NOs:4-19.

12. The peptide of claim 11, wherein the cell importation signal sequence is the sequence of SEQ ID NO:4.

13. The peptide of any one of claims 11-12, wherein the peptide comprises STY-RMR (SEQ ID NO:2).

14. The peptide of any one of claims 11-12, wherein the peptide consists of STY-RMR (SEQ ID NO:2).

15. The peptide of any one of claims 1-14, wherein the peptide is a synthetic peptide.

16. The peptide of any one of claims 1-14, wherein the peptide is a recombinant peptide.

17. The peptide of any one of claims 1-16, wherein the peptide is 25-45 amino acids in length.

18. The peptide of any one of claims 1-16, wherein the peptide is 26-40 amino acids in length.

19. The peptide of any one of claims 1-16, wherein the peptide is 26-36 amino acids in length.

20. The peptide of any one of claims 1-19, wherein the peptide is or consists of SEQ ID NO:1.

21. The peptide of any one of claims 1-20, wherein the peptide is comprised in a pharmaceutical composition.

22. The peptide of claim 21, wherein the pharmaceutical composition is formulated for intravenous, intratumoral, parenteral, intraocular, intracorneal, or intravitreal administration.

23. A fusion protein comprising:
(i) a peptide comprising a region that is at least 90% identical to SEQ ID NO:1, wherein the peptide does not comprise a full-length RTEF-1 polypeptide, an RTEF 669 isoform (SEQ ID NO:20), an RTEF 651 isoform (SEQ ID NO:21), or an RTEF 366 isoform (SEQ ID NO:22); and
(ii) a heterologous amino acid sequence;
wherein the fusion protein can reduce VEGF promoter activity.

24. The fusion protein of claim 23, wherein the peptide comprises less than 121 contiguous amino acids of an RTEF-1 polypeptide.

25. The fusion protein of any one of claims 23-24, wherein the peptide comprises no more than 45 contiguous amino acids of an RTEF-1 polypeptide.

26. The peptide of any one of claims 23-25, wherein the peptide is less than 121 amino acids in length.

27. The fusion protein of claim 26, wherein the peptide is less than 45 amino acids in length.

28. The fusion protein of any one of claims 23-27, wherein the fusion protein is less than 45 amino acids in length.

29. The fusion protein of any one of claims 23-28, wherein the peptide has an amino acid sequence at least 95% identical to SEQ ID NO: 1.

30. The fusion protein of claim 29, wherein the STY peptide has an amino acid sequence at least 97% identical to SEQ ID NO:1.

31. The fusion protein of claim 30, wherein the STY peptide is or consists of the sequence of SEQ ID NO:1.

32. The fusion protein of any one of claims 23-31, wherein the heterologous amino acid sequence is a cell importation signal sequence.

33. The fusion protein of claim 32, wherein the cell importation signal sequence is RMR (SEQ ID NO:4).

34. The fusion protein of any one of claims 23-33, wherein the fusion protein comprises STY-RMR (SEQ ID NO:2).

35. The fusion protein of claim 34, wherein the fusion protein consists of STY-RMR (SEQ ID NO:2).

36. The fusion protein of any one of claims 23-35, wherein the peptide is comprised in a pharmaceutical composition.

37. A composition comprising a peptide comprising a region that is at least 90% identical to SEQ ID NO:1, wherein the peptide does not comprise a full-length RTEF-1 polypeptide, an RTEF 669 isoform (SEQ ID NO:20), an RTEF 651 isoform (SEQ ID NO:21), or an RTEF 366 isoform (SEQ ID NO:22); and wherein the peptide is chemically conjugated to a heterologous amino acid sequence; wherein the composition can reduce VEGF promoter activity.

38. The composition of claim 37, wherein the peptide comprises less than 121 contiguous amino acids of an RTEF-1 polypeptide.

39. The composition of any one of claims 37-38, wherein the peptide comprises no more than 45 contiguous amino acids of an RTEF-1 polypeptide.

40. The composition of any one of claim 37-39, wherein the peptide is less than 121 amino acids in length.

41. The composition of any one of claims 37-40, wherein the peptide is less than 45 amino acids in length.

42. The composition of any one of claims 37-41, wherein the peptide has an amino acid sequence at least 95% identical to SEQ ID NO: 1.

43. The composition of claim 42, wherein the peptide has an amino acid sequence at least 97% identical to SEQ ID NO:1.

44. The composition of claim 43, wherein the peptide is or consists of the sequence of SEQ ID NO:1.

45. The composition of any one of claims 37-44, wherein the heterologous amino acid sequence is a cell importation signal sequence.

46. The composition protein of claim 45, wherein the cell importation signal sequence is RMR (SEQ ID NO:4).

47. The composition of any one of claims 45-46, wherein the peptide comprises STY-RMR (SEQ ID NO:2).

48. The composition of claim 47, wherein the peptide consists of STY-RMR (SEQ ID NO:2).

49. The composition of any one of claims 37-48, wherein the composition is a pharmaceutical composition comprising an excipient.

50. A nucleic acid comprising a nucleic acid segment encoding a peptide or fusion protein of any one of claims 1-36.

51. The nucleic acid of claim 50, wherein the nucleic acid is comprised in a vector.

52. The nucleic acid of claim 51, wherein the vector is a viral vector or a liposome.

53. The nucleic acid of claim 52, wherein the vector is a viral vector that is an adenovirus vector, an adeno-associated virus vector, a herpes virus vector, an SV-40 virus vector, a retrovirus vector, or a vaccinia virus vector.

54. The nucleic acid of any one of claims 50-53, wherein the nucleic acid segment is operatively linked or coupled to a promoter.

55. The nucleic acid of claim 54, wherein the promoter is a cell type specific promoter or an inducible promoter.

56. The nucleic acid of claim 55, wherein the inducible promoter is a hypoxia inducible promoter.

57. The nucleic acid of claim 55, wherein the inducible promoter is an angiogenesis inducible promoter.

58. The nucleic acid of any one of claims 50-57, wherein the nucleic acid encodes two or more antiangiogenesis proteins.

59. A cell comprising the nucleic acid of any one of claims 50-58.

60. The cell of claim 59, wherein the cell is a bacterium, a yeast, an insect cell, or a mammalian cell.

61. A viral vector comprising the nucleic acid of any one of claims 50-58.

62. The viral vector of claim 61, wherein the viral vector is a lentivirus, an adenovirus, or an adeno-associated virus.

63. A method of producing the peptide of any one of claims 1-36 comprising:
a) expressing a nucleic acid of any one of claims 50-58 in a cell; and
b) collecting the peptide or fusion protein therefrom.

64. A method of decreasing angiogenesis in an organism comprising administering to the organism a peptide, fusion protein, or composition of any one of claims 1-49.

65. The method of claim 64, wherein the organism is a mammal.

66. The method of claim 65, wherein the mammal is a human.

67. A method of treating a cancer or an angiogenic eye disease in a mammalian subject, comprising administering to the subject a therapeutically effective amount of a peptide, fusion protein, or composition of any one of claims 1-49.

68. The method of claim 67, wherein the subject is a human, mouse, rat, primate, monkey, or ape.

69. The method of claim 68, wherein the subject is a human.

70. The method of any one of claims 67-69, wherein the subject has a cancer.

71. The method of claim 70, wherein the cancer is a breast cancer, a retinoblastoma, a melanoma, or an ocular cancer.

72. The method of claim 71, wherein the cancer is an ocular cancer selected from the group consisting of an ocular metastasis, an ocular micro-metastasis, or an ocular melanoma.

73. A pharmaceutical composition comprising: the peptide, fusion protein, or composition of any of claims 1-48; and a pharmaceutically acceptable carrier or excipient.

74. The pharmaceutical composition of claim 73, wherein the pharmaceutical composition is formulated for intravenous, intratumoral, parenteral, intraocular, intracorneal, or intravitreal administration.

75. A pharmaceutical composition in accordance with claim 73 for treating a subject with a disorder associated with abnormal cell growth or abnormal cell proliferation.

76. The pharmaceutical composition of claim 75, wherein the disorder associated with abnormal cell growth or abnormal cell proliferation is an angiogenic disorder, a cancer, ocular neovascularization, an arterio-venous malformation, coronary restenosis, peripheral vessel restenosis, glomerulonephritis, or rheumatoid arthritis.

77. The pharmaceutical composition of claim 76, wherein the angiogenic disorder is cancer.

78. The pharmaceutical composition of claim 77, wherein the cancer is breast cancer, lung cancer, prostate cancer, leukemia, lymphoma, head and neck cancer, brain cancer, stomach cancer, intestinal cancer, colorectal cancer, renal cancer, bladder cancer, testicular cancer, esophageal cancer, ocular melanoma, retinoblastoma, liver cancer, ovarian cancer, skin cancer, cancer of the tongue, cancer of the mouth, or metastatic cancer.

79. The pharmaceutical composition of claim 76, wherein the angiogenic disorder is ocular neovascularization.

80. The pharmaceutical composition of claim 79, wherein the ocular neovascularization is neovascularization due to age-related macular degeneration, neovascularization due to corneal graft rejection, neovascularization due to retinopathy of prematurity (ROP), or neovascularization due to diabetic retinopathy.

81. The pharmaceutical composition of claim 76, wherein the subject is further treated with an additional therapy for the disorder.

82. The pharmaceutical composition of claim 81, wherein the additional therapy is an antibody that binds to VEGF, a VEGF receptor, FGF, an FGF receptor, bevacizumab, ranibizumab, or pegaptanib sodium.

83. The pharmaceutical composition of claim 81, wherein the additional therapy is an anticancer therapy that is chemotherapy, surgical therapy, immunotherapy or radiation therapy.

84. The pharmaceutical composition of any one of claims 76-83, wherein the subject is a human.

85. The pharmaceutical composition of any one of claims 76-84, wherein the composition is administered intravenously, intraarterially, epidurally, intrathecally, intraperitoneally, subcutaneously, orally, or topically.

86. The pharmaceutical composition of any one of claims 76-84, wherein the composition is administered locally to the eye by topical drops, intracameral injection, subconjunctival injection, subtenon injection, or by intravitreous injection.

87. Use of a peptide, fusion protein, or composition of any one of claims 1-49 in the manufacture of a medicament for the treatment of a disorder associated with abnormal cell growth or abnormal cell proliferation.

88. A method of treating a disorder associated with abnormal cell growth or abnormal cell proliferation in a subject in need thereof comprising administering a therapeutically effective amount of the pharmaceutical composition of claim 73 to the subject.

89. A kit comprising a predetermined quantity of a peptide, fusion protein, or composition of any of claims 1-49 or a nucleic acid of any of claims 50-58 in one or more sealed vials.


SEQUENCE LISTING (32 sequences)

SEQ ID NO: 1 - protein, 26 aa, Artificial Sequence
  Synthetic Peptide
SSFYGVSSQYESPENMIITCSTKVCS

SEQ ID NO: 2 - protein, 36 aa, Artificial Sequence
  Synthetic Peptide
SSFYGVSSQYESPENMIITCSTKVCSRMRRMRRMRR

SEQ ID NO: 3 - skipped

SEQ ID NO: 4 - protein, 10 aa, Artificial Sequence
  Synthetic Peptide
RMRRMRRMRR

SEQ ID NO: 5 - protein, 10 aa, Artificial Sequence
  Synthetic Peptide
RGRRGRRGRR

SEQ ID NO: 6 - protein, 10 aa, Artificial Sequence
  Synthetic Peptide
RRRRRRRRRR

SEQ ID NO: 7 - protein, 10 aa, Artificial Sequence
  Synthetic Peptide
RARRARRARR

SEQ ID NO: 8 - protein, 10 aa, Artificial Sequence
  Synthetic Peptide
RTRRTRRTRR

SEQ ID NO: 9 - protein, 10 aa, Artificial Sequence
  Synthetic Peptide
RSRRSRRSRR

SEQ ID NO: 10 - protein, 10 aa, Artificial Sequence
  Synthetic Peptide
RVRRVRRVRR

SEQ ID NO: 11 - protein, 10 aa, Artificial Sequence
  Synthetic Peptide
RKRRKRRKRR

SEQ ID NO: 12 - protein, 7 aa, Artificial Sequence
  Synthetic Peptide
RRRRRRR

SEQ ID NO: 13 - protein, 8 aa, Artificial Sequence
  Synthetic Peptide
RRRRRRRR

SEQ ID NO: 14 - protein, 9 aa, Artificial Sequence
  Synthetic Peptide
RRRRRRRRR

SEQ ID NO: 15 - protein, 11 aa, Artificial Sequence
  Synthetic Peptide
RRRRRRRRRRR

SEQ ID NO: 16 - protein, 12 aa, Artificial Sequence
  Synthetic Peptide
RRRRRRRRRRRR

SEQ ID NO: 17 - protein, 13 aa, Artificial Sequence
  Synthetic Peptide
RRRRRRRRRRRRR

SEQ ID NO: 18 - protein, 14 aa, Artificial Sequence
  Synthetic Peptide
RRRRRRRRRRRRRR

SEQ ID NO: 19 - protein, 15 aa, Artificial Sequence
  Synthetic Peptide
RRRRRRRRRRRRRRR

SEQ ID NO: 20 - protein, 222 aa, Homo sapiens
MEGTAGTITSKLWMLEFSAFLEQQQDPDTYNKHLFVHIGQSSPSYSDPYLEAVDIRQIYDKFPEKKGGLKDLFERGPSNAFFLVKFWADLNTNIEDEGSSFYGVSSQYESPENMIITCSTKVCSFGKQVVEKVETEYARYENGHYSYRIHRSPLCEYMINFIHKLKHLPEKYMMNSVLENFTILQVVTNRDTQETLLCIAYVFEVSASEHGAQHHIYRLVKE

SEQ ID NO: 21 - protein, 216 aa, Homo sapiens
MEGTAGTITSNEWSSPTSPEGSTASGGSQALDKPIDNDAEGVWSPDIEQSFQEALAIYPPCGRRKIILSDEGKMYGRNELIARYIKLRTGKTSSFYGVSSQYESPENMIITCSTKVCSFGKQVVEKVETEYARYENGHYSYRIHRSPLCEYMINFIHKLKHLPEKYMMNSVLENFTILQVVTNRDTQETLLCIAYVFEVSASEHGAQHHIYRLVKE

SEQ ID NO: 22 - protein, 121 aa, Homo sapiens
MEGTAGTITPENMIITCSTKVCSFGKQVVEKVETEYARYENGHYSYRIHRSPLCEYMINFIHKLKHLPEKYMMNSVLENFTILQVVTNRDTQETLLCIAYVFEVSASEHGAQHHIYRLVKE

SEQ ID NO: 23 - protein, 13 aa, Artificial Sequence
  Synthetic Peptide
GRKKRRQRRRPPQ

SEQ ID NO: 24 - protein, 9 aa, Artificial Sequence
  Synthetic Peptide
RKKRRQRRR

SEQ ID NO: 25 - protein, 16 aa, Artificial Sequence
  Synthetic Peptide
RQPKIWFPNRRKPWKK

SEQ ID NO: 26 - protein, 16 aa, Artificial Sequence
  Synthetic Peptide
RQIKIWFQNRRMKWKK

SEQ ID NO: 27 - protein, 26 aa, Artificial Sequence
  Synthetic Peptide
GIGAVLKVLTTGLPALISWIKRKRQQ

SEQ ID NO: 28 - protein, 10 aa, Artificial Sequence
  Synthetic Peptide
TKIESLKEHG

SEQ ID NO: 29 - protein, 10 aa, Artificial Sequence
  Synthetic Peptide
TQIENLKEKG

SEQ ID NO: 30 - protein, 26 aa, Artificial Sequence
  Synthetic Peptide
AALEALAEALEALAEALEALAEAAAA

SEQ ID NO: 31 - protein, 25 aa, Artificial Sequence
  Synthetic Peptide
GLFEAIEGFIENGWEGMIEGWYGCG

SEQ ID NO: 32 - protein, 433 aa, Homo sapiens
LEGTAGTITSNEWSSPTSPEGSTASGGSQALDKPIDNDGEGVWSPDIEQSFQEALAIYPPCGRRKIILSDEGKMYGRNELIARYIKLRTGKTRTRKQVSSHIQVLARRKAREIQAKLKDQAAKDKALQSMAAMSSAQIISATAFHSSMRLARGPGRPAVSGFWQGALPGQAETSHDVKPFSQQTYAQPPLPLPGFESPAGPAPSPSAPPAPPWQGRRRGSSKLWMLEFSAFLEQQQDPDTYNKHLFVHIGQSSPSYLRPYLEAVDIRQIYDKFPEKKGGLKDLFERGPSNAFFLVKFWADLNTNIEDEGSSFYGVSSQYESPENMIITCSTKVCSFGKQVVEKVETEYARYENGHYSYRIHRSPLCEYMINFIHKLKHLPEKYMMNSVLENFTILQVVTNRDTQETLLCIAYVFEVSASEHGAQHHIYRLVKE